(19) United States
(12) Patent Application Publication
(10) Pub. No.: US 2020/0330356 A1
(43) Pub. Date: Oct. 22, 2020

(54) COSMETIC COMPOSITION COMPRISING NANOCRYSTALLINE CELLULOSE, METHOD AND USE THEREOF

(71) Applicant: CHANEL PARFUMS BEAUTE, Neuilly Sur Seine (FR)

(72) Inventor: Philippe GUARILLOFF, Princeton, NJ (US)

(73) Assignee: CHANEL PARFUMS BEAUTE, Neuilly Sur Seine (FR)

(21) Appl. No.: 16/464,382

(22) PCT Filed: Nov. 30, 2017
(86) PCT No.: PCT/EP2017/080989

§ 371 Date: May 28, 2019

Related U.S. Application Data
(60) Provisional application No. 62/427,914, filed on Nov. 30, 2016.

(51) Int. Cl. A61K 8/73; A61K 8/06; A61Q 3/02; A61Q 19/00; A61Q 1/12; A61Q 1/06; A61Q 1/10

(52) U.S. Cl. CPC A61K 8/731; A61K 8/062; A61K 8/064; A61Q 1/10; A61Q 19/00; A61Q 1/12; A61Q 1/06; A61Q 3/02

(57) ABSTRACT

A cosmetic composition including, in a physiologically acceptable medium, nanocrystalline cellulose, wherein the composition is topically applied onto the skin of a subject in need of the application to reduce the visibility of skin imperfections and/or to impart a matte effect on the skin. Also, the use of a nanocrystalline cellulose to provide soft-focus, mattity and natural appearance to the skin.

### BACKGROUND OF THE INVENTION

### Technical Field

The present invention relates to the field of care and/or make-up compositions for the skin. More specifically, the invention relates to cosmetic compositions comprising nanocrystalline cellulose for improving the appearance of the skin, particularly intended to hide skin imperfections by providing mattity and a soft-focus effect to the skin, while retaining a natural appearance.

### Related Prior Art

There is an increasing demand in the cosmetic industry to develop products that provide a flawless look, with no apparent imperfections. Facial skin may be marked by the presence of wrinkles, fine lines, hyperpigmentation, but also by the presence of dilated and visible pores.

Pore size may be determined by genetic, environmental and/or physiological factors. Visible pores are frequently associated with increased sebaceous glands activity. For users having a greasy skin, it is thus desirable not only to reduce the visibility of pores, but also to have a matte finish in order to overcome the shiny effect engendered by the excess of sebum secretion.

A wide variety of methods intended for improving the appearance of the skin have been developed. It is known practice in the art to introduce solid particles into care or makeup compositions to obtain mattifying effect. These particles are generally selected according to their good properties of sebum absorption. Sebum absorbing fillers include for example silica beads which are commonly used in cosmetic compositions. However, these compositions are often accompanied by unpleasant dryness sensations and discomfort due to moisture absorption of the skin.

Another mean of visually reducing skin defects consists in modifying the optical properties at the surface of the skin, by depositing a film of a suitable cosmetic composition, which gives the consumer an immediate visual improvement. When the light hits the film at the surface of the skin, it separates into two components: a transmitted component and a reflected component. The amount of light that is not reflected or transmitted is absorbed by the film of cosmetic composition. Both transmitted and reflected components are divided into a specular component and a diffuse component. In specular reflectance/transmittance, incident light is reflected/transmitted from a surface at a single angle in accordance with the Law of Reflection/Snell's Law respectively. When the light is not reflected nor transmitted at a single angle but scattered in many outgoing directions, the light is considered as diffused.

It is common practice to introduce pulverulent materials in order to obtain a relief filling effect on the skin. These particles interfere with light in various manners, depending on their size, shape and chemical nature. The optical reduction of wrinkles and pores is due to the light diffusing properties of the applied particles. Particles that scatter and thus, diffuse light away, minimize the visibility of skin relief irregularities such as pores, which appear blurred, hence the terms “soft-focus effect” or “blurring effect”. Soft-focus particles should also have a refractive index that is different from that of the medium in which the soft-focus fillers are present. However, if the refractive index of the particle is too high compared to that of the medium, it may lead to an unnatural look on skin owing to the use of highly opaque material.

Moreover, consumers are increasingly looking for makeup compositions, particularly foundations, exhibiting a natural makeup result. It is found, however, that providing a pigmented composition with both soft-focus and mattifying effect can create unnatural caked-on appearance. A covering cosmetic that is too opaque hides the skin transparency under a paint-like coating: imperfections are hidden but the skin appears dull and aesthetically displeasing.

A challenge which has not been fully met by the known art is delivery of a composition with appropriate optics to achieve both blurring and matte effects in a system that still provides natural and smooth appearance to the skin and excellent skinfeel.

### Aims and Advantages of the Invention

The inventors have discovered that the incorporation of nanocrystalline cellulose, also referred to as NCC, in cosmetic compositions allows to overcome the abovementioned drawbacks of the prior art. These particles are present in the compositions in levels that provide an immediate visual improvement of the skin appearance. It is believed that this improvement results at least in part in the optical properties of the nanocrystalline cellulose.

When the compositions of the present invention are applied on the skin, the presence of nanocrystalline cellulose generates an increase of diffuse reflectance, and a low specular reflectance at the surface of the skin, as well as a high diffuse transmittance of the light through the film of cosmetic composition applied on the skin. The skin is flawless while appearing natural.

The combination of the improvement of light backscattering and diffuse transmittance creates a composition that has very good optical benefits, namely the inclusion of nanocrystalline cellulose particles in cosmetic compositions confers immediate matte and soft-focus effects to the skin by reducing the difference in luminosity between the valley and the edges of skin relief.

Thus, one aim of the present invention is to allow reducing skin defects via optical effects.

Another aim of the present invention is to exhibit a higher diffuse reflectance component compared to specular reflectance component, which provides an immediate matte effect to the skin. At the same time, the compositions according to the present invention do not produce an undesirable whitish sheen or film on the skin.

Another aim of the present invention is to exhibit a high diffuse transmittance also referred to as “haze”, for providing a good soft-focus effect.

Another aim of the present invention is to exhibit a high total transmittance value, and thus, contribute forming a translucent film on the skin which gives a natural appearance while improving its uniformity.

### SUMMARY OF THE INVENTION

The present invention is therefore directed to a cosmetic composition comprising, in a physiological acceptable medium, a content of nanocrystalline cellulose.

Accordingly, the present invention relates to a cosmetic composition having a haze, (T_{d}/Tₜₒₜ)*100, of greater than or equal to 50%, and/or a total transmittance, Tₜₒₜ, of greater than or equal to 70%, and/or a mattity, (R_{d}/Rₜₒₜ)*100, of greater than or equal to 70%, and/or a diffuse reflectance, Rd, of lower than or equal to 10%.

Another object of the invention is to provide a cosmetic composition comprising nanocrystalline cellulose, wherein said nanocrystalline cellulose is a functionalized nanocrystalline cellulose.

Another object of the invention is to provide a cosmetic composition comprising functionalized nanocrystalline cellulose, wherein said functionalized nanocrystalline cellulose is carboxylated nanocrystalline cellulose.

Another object of the invention is to provide a cosmetic composition comprising carboxylated nanocrystalline cellulose, wherein said carboxylated nanocrystalline cellulose is in the form of a nanocrystalline cellulose carboxylate salt.

Another object of the invention is to provide a cosmetic composition comprising nanocrystalline cellulose carboxylate salt, wherein said nanocrystalline cellulose carboxylate salt is a nanocrystalline cellulose sodium carboxylate.

Another object of the invention is to provide a cosmetic composition comprising nanocrystalline cellulose sodium carboxylate, wherein said nanocrystalline cellulose sodium carboxylate is produced by the method comprising the steps of:
a) providing cellulose,
b) mixing said cellulose with a peroxide, thereby producing a reaction mixture,
c) heating the reaction mixture, and/or exposing the reaction mixture to UV radiation, and
d) salifying the reaction mixture.

Another object of the invention is to provide a cosmetic composition comprising nanocrystalline cellulose, wherein said nanocrystalline cellulose has a spherical or ovoid shape.

Another object of the invention is to provide a cosmetic composition comprising nanocrystalline cellulose, wherein said nanocrystalline cellulose has an average particle size of less than about 20 μm, preferably from 2 μm to 10 μm. The average particle size is the particle size distribution D50, also known as the median diameter or the medium value of the particle size distribution, it is the value of the particle diameter at 50% in the cumulative distribution. The particle size distribution is determined by laser diffraction granulometry.

Another object of the invention is to provide a cosmetic composition comprising nanocrystalline cellulose, wherein said nanocrystalline cellulose has a refractive index comprised between 1.35 and 1.6. Another object of the invention is to provide a cosmetic composition comprising nanocrystalline cellulose, and further comprising at least one coloring agent selected from pigments and/or nacres.

Another object of the invention is to provide a cosmetic composition comprising nanocrystalline cellulose and further comprising at least one filler selected from organic fillers and/or inorganic fillers.

Another object of the invention is to provide a cosmetic composition comprising nanocrystalline cellulose and further comprising at least one aqueous phase.

Another object of the invention is to provide a cosmetic composition comprising nanocrystalline cellulose and further comprising at least one fatty phase

Another object of the invention is to provide a cosmetic composition comprising nanocrystalline cellulose and further comprising at least one UV screening agent selected from mineral UV screening agents and/or organic UV screening agents.

Another object of the invention is to provide a cosmetic composition comprising nanocrystalline cellulose and further comprising at least one additional ingredient selected from preservatives, cosmetic active ingredients, moisturizers, surfactants and/or fragrances.

The compositions according to the invention may be a in the form of a water-in-oil emulsion or an oil-in-water emulsion, a compact powder, a loose powder, an anhydrous fluid, a cream, a lotion, a stick, a hot poured composition, and the like. The compositions according to the invention may be pigmented or not pigmented. The compositions according to the invention may be selected from face powders such as foundations, concealers, blushers, highlighters, makeup bases, skin care compositions, and the like. The compositions according to the invention are also suitable for achieving deeper ethnic skin tones where the use of white fillers can tend to give the user an undesirable ashy and/or dull appearance. The compositions according to the invention may also be in the form of eye shadows, lipsticks, lip glosses, nail enamels, and the like.

According to one preferred embodiment, the composition of the present invention comprises pigments.

According to another preferred embodiment, the composition of the present invention is a makeup composition.

According to another preferred embodiment, the composition of the present invention is a foundation, a makeup base or a concealer.

Other characteristics, aspects and advantages of the present invention will become apparent on reading the detailed description which follows.

### DETAILED DESCRIPTION

The cosmetic compositions according to the present invention comprise nanocrystalline cellulose, also referred to as “NCC”. Nanocrystalline cellulose is derived from native cellulose from which the amorphous part is removed to keep only the crystalline part.

According to the present invention, the amorphous part of native cellulose is advantageously removed by oxidative hydrolysis of native cellulose using a peroxide, such as hydrogen peroxide, an organic peroxide or a mixture thereof. This process of dissolution of the amorphous part of native cellulose using a peroxide produces nano-crystallites of cellulose, which are then assembled into larger particles corresponding to said nanocrystalline cellulose or NCC.

According to a first embodiment of the present invention, said nanocrystalline cellulose is functionalized, i.e. it has undergone a surface modification to produce functionalized nanocrystalline cellulose. According to a preferred embodiment, said functionalized nanocrystalline cellulose is a carboxylated nanocrystalline cellulose.

Advantageously, carboxylated nanocrystalline cellulose may undergo total or partial salification to produce nanocrystalline cellulose carboxylate salt.

According to another embodiment, the nanocrystalline cellulose carboxylate salt according to the present invention is produced by the method comprising the steps of:

a) providing cellulose.

b) mixing said cellulose with a peroxide, thereby producing a reaction mixture,

c) heating the reaction mixture, and/or exposing the reaction mixture to UV radiation, and

d) salifying the reaction mixture.

According to another embodiment, the assemblage of nano-crystallites of cellulose into particles of nanocrystalline cellulose is achieved by spray-drying.

According to another embodiment, said particles of nanocrystalline cellulose have a spherical or ovoid shape, or a mixture thereof.

According to another embodiment, nanocrystalline cellulose has an average particle size of less than 20 μm, preferably less than 15 μm, more preferably between 2 μm and 10 μm.

According to another embodiment, nanocrystalline cellulose has an oil uptake of less than 60 mL/100 g, preferably between 45 and 55 mL/100 g. The oil uptake characterizes the ability to adsorb castor oil. It is determined by adding castor oil to 100 g of nanocrystalline cellulose powder. The oil uptake corresponds to the minimal amount of castor oil, in milliliters, required to obtain a firm and homogeneous paste.

Another to another embodiment, nanocrystalline cellulose has a refractive index comprised between 1.35 and 1.6, preferably between 1.4 and 1.6. The refractive index is determined by immersion method. A few milligrams of powder are added to a series of liquids of known refractive index. The particles of nanocrystalline cellulose become invisible in a liquid with the same refractive index. The mixtures were observed under Olympus BX50 microscope.

According to another embodiment, nanocrystalline cellulose has a contact angle with water between 80° and 100°, preferably between 85° and 95°, and more preferably between 88° and 92°.

Other possible embodiments of nanocrystalline cellulose in the compositions of the invention are included in the disclosure of the PCT patent application WO 2016/015148 from The Royal Institution for the Advancement of Learning/McGill University, incorporated herein by reference.

The compositions according to the invention are intended to minimize or decrease the visibility of skin imperfections, in particular pores, fine lines and wrinkles. In accordance with the previous embodiments, the compositions of the present invention are applied topically to the desired area in an amount sufficient to optically minimize the visibility of skin imperfections. When a composition of the present invention is applied onto the skin, the composition has an homogenizing effect on skin, that is, the composition produces a blurring/haze or soft-focus effect on the skin as well as an immediate matte effect. The visibility of skin imperfections such as pores, wrinkles and fine lines are minimized. At the same time, the composition of the present invention does not produce an undesirable whitish sheen or film on the skin, but is transparent/translucent such that the skin has a natural appearance.

The haze, (T_{d}/Tₜₒₜ)*100, may be measured according to the protocol described below using a spectrophotometer and an integration sphere, for example placed behind the sample.
T_{d} is the diffuse transmittance of the composition: it is the ratio of the diffusely transmitted radiant power to incident radiant power (specular component is excluded).
Tₜₒₜ is the total transmittance of the composition: it is defined by the ratio of the total transmitted radiant power (diffuse and specular) to incident radiant power.

The mattity, (R_{d}/Rₜₒₜ)*100, may also be measured by spectrophotometer with an integration sphere.
R_{d} is the diffuse reflectance of the composition: it is the ratio of the diffusely reflected radiant power to incident radiant power (specular component is excluded).
Rₜₒₜ is the total reflectance of the composition: it is defined by the ratio of the total reflected radiant power (diffuse and specular) to incident radiant power.

In vitro optical measurements defined above are obtained using a Konica Minolta CM-3600A spectrophotometer comprising an integration sphere disposed behind or ahead of the sample based on the following protocol. An ERICHSEN automatic draw-down instrument was used to produce 30 μm-thick films of the compositions on 100 μm-thick PET substrates from BYK sold under reference No. 2870. The films were dried for four hours at 35° C. The spectrophotometer is used after calibration in diffuse transmission mode to measure T_{d} and it is used it total transmission mode to measure Tₜₒₜ. Reflectance values may be measured with the same spectrophotometer in diffuse reflection mode and total reflection mode for measuring R_{d} and Rₜₒₜ respectively. In all case, the machine is set in % mode for transmission and reflection modes, at a wavelength ranging from 400 to 700 nm.

A higher value of mattity indicates a better immediate matte effect. A higher total transmittance value indicates a better transparent film of composition on the skin. A higher value of haze indicates a better blurring effect. A lower diffuse reflectance value indicates a low white effect on the skin.

As disclosed herein, the haze value of the composition according to the invention is greater than 50%, preferably greater than or equal to 60%, more preferably greater than or equal to 75%.

The total transmittance, here also referred to as “transparency index”, may be greater than or equal to 70%, preferably greater than or equal to 80%, more preferably greater than or equal to 85%.

The mattity may be, for example be greater than or equal to 70%, preferably greater than or equal to 75%, more preferably greater than or equal to 85%.

The diffuse reflectance, here also referred to as “whitening index” may be lower than or equal to 10%. Whitening is a measure of white cast that appears on the skin after the product is applied, and is undesirable.

As disclosed herein, the composition according to the present invention comprises an nanocrystalline cellulose allowing to obtain such haze, mattity, transparency index, and whitening index defined above.

The compositions according to the inventions may also comprise at least one humectant. Non-limiting example include glycerol derivatives such as glycerin, butylene glycol, propylene glycol, caprylyl glycol; urea derivatives; lactic acid derivatives.

The compositions according to the invention may comprise at least one oily phase, which may comprise at least one liquid oil.

The term “liquid” refers to compounds in a liquid state at room temperature (i.e. 20° C.) and atmospheric pressure (i.e. 1,013×105 Pa).

The term “oil” refers to any compound that is not miscible in water and which is liquid at room temperature (i.e. 20° C.) and atmospheric pressure (i.e. 1,013×105 Pa).

The liquid binder phase advantageously includes at least one non-volatile oil, which may be hydrocarbon-based oil, silicone-based oil or a mixture thereof. The oils according to the invention may be synthetic or from natural origin.

The term “non-volatile oil” is understood to mean any liquid oil which is not capable of evaporating on contact with the skin, and thus remaining on the skin.

The term “hydrocarbon-based oils” means oils mainly containing carbon atoms and hydrogen atoms, and which may also comprise one or more functional group selected from alcohol, ether, ester, fluoro and/or carboxylic acid groups.

The term “silicone-based oils” means oils containing silicon atoms but also oxygen, carbon and hydrogen atoms. Silicone-based oils may also comprise one or more functional group such as alcohol, ether, ester, fluoro and/or carboxylic acid groups.
Silicone-based oils include but are not limited to linear and cyclic non-volatile polydimethylsiloxanes, polymethylphenylsiloxanes, phenyl dimethicones, phenyl trimethicones; polysiloxanes modified with fatty acids fatty alcohols, alkylene oxyalkylene groups or, amine group; fluorosilicones or perfluoro silicone oils;
Hydrocarbon-based oils include hydrocarbon oils, esters of fatty acids, fatty alcohols, fatty acids and/or vegetable oils.
Hydrocarbon oils which may be linear or branched, saturated or unsaturated, such as liquid paraffins, mineral oil, squalane, squalene, polydecenes, polybutenes and derivatives;
Esters of fatty acids of general formula R₁COOR₂ wherein R₁ represents a linear or branched fatty acid residue containing from 1 to 40 carbon atoms, preferably from 1 to 30 carbon atoms, more preferably from 1 to 22 carbon atoms, and R₂ represents a hydrocarbon-based chain which may be linear or branched too, and containing from 1 to 40 carbon atoms. These two carbon chains may be saturated or unsaturated. The esters may also contain a polyalkylene glycol branching such as polypropylene glycol or polyethylene glycol branching, for example PPG-2 myristyl ether propionate. The compositions according to the invention may also comprise polyesters, i.e. compounds comprising more than one ester functional group such as diesters or triesters. Mention may be made of triglycerides formed by esterification of glycerol such as caprylic/capric triglyceride; esters of polyglycerin such as polyglyceryl-2 triisostearate; triethylhexanoin, dicaprylyl carbonate or octyldodecyl stearoyl stearate. The acid residue may also be cyclic, such as in esters of benzoic acid or esters of salicylic acid.

Suitable fatty acid esters include without limitation isononyl isononanoate, isopropyl myristate, 2 ethylhexyl palmitate, hexyl laurate, diisostearyl malate, C12-15 Alkyl Ethylhexanoate, cetyl ethylhexanoate, octyl stearate, isodecyl neopentanoate, isostearyl palmitate, alkyl benzoates, butyl acetate, butyl lsostearale, butyl oleate, butyl octyl oleate, cetyl palmilale, ceyl oclanoale, celyl laurate, cetyl lactate, cetyl isononanoate, cetyl stearate, diisostearyl fumarate, diisostearyl malale, neopentyl glycol dioctanoate, dibutyl sebacate, di-C₁₂₋₁₃ alkyl malate, dicetearyl dimer dihnoleate, dicetyl adipate, dusocetyl adipate, dusononyl adipate, dusopropyl dunerate, triisostearyl trihnoleate, octodecyl stearoyl stearate, hexyl laurate, hexadecyl isostearate, hexydecyl laurate, hexyldecyl octanoate, hexyldecyl oleate, hexyldecyl palmitate, hexyldecyl stearate, isononyl isononanaote, isostearyl lsononate, isohexyl neopentanoate, isohexadecyl stearate, isopropyl isostearate, n-propyl myristate, isopropyl mynstate, n-propyl palmitate, isopropyl palmitate, hexacosanyl palmitate, lauryl lactate, octacosanyl palmitate, propylene glycol monolaurate, triacontanyl palmitate, dotriacontanyl palmitate, tetratriacontanyl palmitate, hexacosanyl stearate, octacosanyl stearate, triacontanyl stearate, dotriacontanyl stearate, stearyl lactate, stearyl octanoate, stearyl heptanoate, stearyl stearate, tetratriacontanyl stearate, triarachidin, tributyl citrate, triisostearyl citrate, tri-C[12-13]-alkyl citrate, tricapryhn, tricaprylyl citrate, tridecyl behenate, trioctyldodecyl citrate, tridecyl cocoate, tridecyl isononanoate, glyceryl monoricinoleate, 2-octyldecyl palmitate, 2-octyldodecyl myristate or lactate, di(2-ethylhexyl) succinate, tocopheryl acetate, tripropylene glycol dineopentanoate, cetyl octanoate, cetyl isooctanoate, octyldodecyl myristate, isopropyl palmitate, cetyl palmitate, butyl stearate, hexyl laurate, myristyl myristate, decyl oleate, hexyldecyl dimethyloctanoate, cetyl lactate, myristyl lactate, acetylated lanolin, isocetyl stearate, isocetyl isostearate, cholesteryl 12-hydroxystearate, ethylene glycol di-2-ethylhexanoate, dipentaerythritol fatty acid ester, N-alkylglycol monoisostearate, neopentyl glycol dicaprylate, glyceryl di-2-heptylundecanoate, trimethylolpropane tri-2-ethylhexanoate, trimethylolpropane triisostearate, pentaerythritol tetra-2-ethylhexanoate, glyceryl tri-2-ethylhexanoate, glyceryl trioctanoate, glyceryl triisopalmitate, trimethylolpropane triisostearate, cetyl 2-ethylhexanoate, glyceryl trimyristate, glyceride tri-2-heptylundecanoate, castor oil fatty acid methyl ester, oleyl oleate, acetoglyceride, 2-heptylundecyl palmitate, diisobutyl adipate, N-lauroyl-L-glutamic acid-2-octyldodecyl ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, triethyl citrate, bis-behenyllisostearyl/phytosteryl dimer dilinoleyl dimer dilinoleate, phytosteryl/behenyl/octyldodecyl/isostearyl lauroyl glutamate, caprylic/capric triglyceride, and triethylhexanoin.
Fatty alcohols, preferably having from 5 to 40 carbon atoms such as octyldodecanol and oleyl alcohol.
Fatty acids preferably having from 5 to 40 carbon atoms such as linoleic or linolenic acid.
Vegetable oils and derivatives, such as soybean oil, jojoba oil, olive oil, macadamia oil, liquid butyrospermum parkii (shea butter), castor oil, camellia oil, gardenia oil, avocado oil, coconut oil, argania spinosa kernel oil, corn oil, cottonseed oil, linseed oil, mink oil, soybean oil, grape seed oil, sesame oil, maize oil, rapeseed oil, sunflower oil, peanut oil, teas seed oil, rice bran oil.
Sarcosine derivatives such as isopropyl lauroyl sarcosinate.

The composition advantageously comprises at least one volatile oil.

The cosmetic oils that are volatile at ambient temperature especially have a vapour pressure, measured at ambient temperature and atmospheric pressure, ranging from 10-3 mmHg to 300 mmHg (0.266 Pa to 40 000 Pa).

As a volatile oil that can be used in the invention, mention may be made of linear or cyclic silicones oils that have a viscosity at ambient temperature of less than 8 cSt and that especially have from 2 to 7 silicon atoms, these silicones optionally comprising alkyl or alkoxy groups having from 1 to 10 carbon atoms. As a volatile silicone oil that can be used in the invention, mention may especially be made of octamethylcyclotetrasiloxane, decamethylcyclopentasiloxane, dodecamethylcyclo¬hexa-siloxane, heptamethylhexyltrisiloxane, heptamethyl-octyl¬trisiloxane, hexamethyldisiloxane, octamethyl-trisiloxane, decamethyltetrasiloxane, dodecamethylpentasiloxane and mixtures thereof.

As another volatile oil that can be used in the invention, mention may be made of volatile hydrocarbon-based oils having from 8 to 16 carbon atoms and mixtures thereof, and especially C8-C16 branched alkanes such as C8-C16 isoalkanes (also called isoparaffins), isododecane, isodecane, isohexadecane and, linear volatile alkanes from 9 to 14 carbon atoms, such as those sold under the names Vegelight 1214 from Biosynthis and Cetiol Ultimate from BASF.

The compositions according to the invention may also comprise at wax.

The waxes that are suitable for the present invention have a melting point is comprised in the range from 30° C. and 200° C. The compounds that may be used in the present invention include natural waxes such as those of animal origin, vegetable origin or mineral origin, and synthetic waxes.

Non-limiting example of waxes that may be used in the present invention include but are not limited to beeswax, lanolin wax and derivatives, jojoba wax, shellac wax, carnauba wax, candelilla wax, castor wax, bayberry wax, soy wax, hardened coconut oil, palm kernel oil, cacao butter, polycosanols, ozokerite wax, ceresin wax, paraffin waxes, microcrystalline waxes, vaseline, Fischer-Tropsch waxes, polyolefin waxes such as polyethylene wax, polyethylene glycol wax, hydrogenated polyisobutene, and mixture thereof. Mention may also be made of fatty acid esters or diesters solid at room temperature, such as Stearyl/PPG-3 Myristyl ether dimer dilinoleate or isostearyl hydroxystearate; silicone waxes such as C24-28 alkyl methicone or stearoxymethicone & dimethicone copolymer; micronized waxes, i.e. waxes in powder form, such as polypropylene micronized wax, carnauba wax microbeads, and mixtures thereof.

The compositions according to the invention may also comprise organopolysiloxane elastomers, which may be emulsifying silicone elastomers or non-emulsifying elastomers. Examples of such elastomers are especially sold by SHIN ETSU under the trade names KSG-6, KSG-16, KSG-31, KSG-32, KSG-41, KSG-42, KSG-43, KSG-44 KSG-21 and KSG-210, by DOW CORNING under the trade names DC 9040 and DC 9041 and by GRANT INDUSTRIES under the trade name Gransil.

The compositions according to the invention may also comprise at least one film former polymer, which may be chosen from silicone film former and/or non-silicone film former.

Among silicone film former polymers we can mention:
Silicone resins as for example MQ-resins, T-resins or MTQ resins, such as Trimethylsiloxysilicate sold under commercial name Belsil TMS 803 sold by Wacker Chemie AG, Trimethylsiloxysilicate & polypropylsilsesquioxane sold under commercial name MQ1640 Flake Resin by Dow Corning,
Silicone acrylates as for example Isododecane & acrylates/polytrimethylsiloxymethacrylate copolymer sold under commercial name Dow Corning® FA4002 ID silicone acrylate by Dow Corning,
Silicone Pullulan film-formers such as Trimethylsiloxysilylcarbamoyl Pullulan sold under commercial name TSPL-30-ID-F by Shin-Etsu Chemical Co,
Silicone Pressure Sensitive Adhesive, such as Dimethicone & trimethylsiloxysilicate/dimethiconol crosspolymer sold under commercial name Dow Corning® 7-4411 Cosmetic Fluid by Dow Corning,
High molecular weight branched polymer, such as DOW CORNING® 3901 Liquid Satin Blend sold by Dow Corning. Its INCI name is Dimethicone (and) Dimethicone/Vinyl Dimethicone Crosspolymer. Its rheological behavior is unique and is different from silicone elastomers, of which some have the same INCI name. In particular it exhibits a pituitous fluid behavior.
Fluorated silicone resins such as Dimethicone & Trifluoropropyldimethyl/rrimethylsiloxysilicate sold under commercial name XS66-B8636 by Shin Etsu,
Silicone urethane film-former such as Bis-Hydroxypropyl Dimethicone/SMDI copolymer sold under commercial name Silmer UR 5050 by Siltech LLC,
Phenyl trimethicone & diphenyl dimethicone crosspolymer (developped by Nusil under the name CXG-1106) or propanediol dicaprylate & diphenyl dimethicone crosspolymer (developped by Nusil under the name CXG-1108)
Aqueous dispersion of encapsulated silicone elastomer, such as CES-1104 sold by NuSil (INCI name: Dimethicone (and) Water (and) Glycerin (and) Pentylene Glycol (and) Dimethicone/Vinyl Dimethicone Crosspolymer (and) Amodimethicone (and) Carbomer (and) Phenoxyethanol (and) Sodium Hydroxide (and) Disodium EDTA)
Aqueous dispersion of encapsulated fluoro silicon derivative such CES-3401 sold by Nusil (INCI name: Trifluoropropyl Dimethicone & Aqua & Glycerin & pentylene Glycol & Amodimethicone & Carbomer & Sodium Hydroxide & Phenoxyethanol & Disodium EDTA).

Among non-silicone film former polymers we can mention:
Sucrose acetate isobutyrate sold under commercial name Sustane SAIB by Eastman Chemical Company.
Green polyesters sold by Surfatech under commercial name COSMOSURF DDG-20 (Octyldodecyl/propane diol/dimer dilinoleate copolymer) or DDG-28 (Dodecylhexadecyl/propane diol/dimer dilinoleate copolymer).

According to one embodiment, the compositions of the invention may comprise at least one surfactant. As emulsifying surfactants that may be used, mention may be made of sorbitan, glycerol or sugar alkyl esters or ethers, such as polyglyceryl isostearate, sorbitan isostearate, polysorbate-65; silicone surfactants such as dimethicone copolyol and alky dimethicone copolyol such as lauryl dimethicone.

The compositions according to the invention may also comprise powders, which may be chosen from organic powders and/or inorganic powders.

The powders used in the compositions according to the present invention may also be hollow particles, porous particles or non-porous particles. They may be surface-coated or not.

Among the surface-coatings that may be used in the present invention, mention may be made of aluminium hydroxide; alumina; polyurethane derivatives; polyquaternium derivatives; silicone derivatives such as triethoxycaprilylsilane (OTS coating from Daito Kasei), triethoxysilylethyl polydimethylsiloxyethylhexyl dimethicone, acrylates/dimethicone copolymer (FSA coating from Daito Kasei), methicone or dimethicone; amino-acid derivatives or N-acylamino acids or salts thereof such as sodium lauroyl glutamate, sodium lauroyl aspartate, lysine, disodium stearoyl glutamate, lauroyl lysine; fluoro derivatives such as perfluoroalkylsilanes, perfluoroalkylsilazanes, perfluoroalkyl phosphates, C9-C15 fluoroalcohol phosphates; lecithin derivatives such as hydrogenated lecithin; alkyl titanated derivatives such as isopropyl titanium triisostearate; silica; silicates such as potassium aluminium silicate; fatty acid derivative such as stearic acid; metallic soaps such as aluminium dimyristate, aluminium stearate, magnesium myristate, metal oxides such as titanium dioxide, zinc oxide or iron oxide; and mixture thereof.

The compositions according to the present invention may include at least one filler different from nanocrystalline cellulose. The term “filler” as used herein means a white or colorless solid particle, which is intended to give texture and body to cosmetic compositions. The fillers may also confer softness, matity and uniformity to the cosmetic compositions.

A composition according to the present invention may advantageously have a content of fillers of at least 0.1% by weight relative to the total composition, preferably from 1% to 90% by weight, more preferably from 3% to 80% by weight.

The fillers which may be used may have a spherical, lamellar, ovoid, or globular shape or being in the form of fibers. They may also be in any intermediate shape between these defined shapes.

talc, boron nitride, mica, synthetic fluorphlogopite, hydroxyapatite, alumina, silk powder, pearl powder, barium sulfate powder, cellulose powder, microcrystalline cellulose powder, perlite, glass, ceramic; clays such as muscovite, phlogopite, kaolin, hectorite or bentonite; silica based-materials such as silica, fumed silica, or silica silylate; quartz, or gemstones such as gold or diamond powders.

The inorganic fillers that may also be used in the compositions according to the invention may also be chosen from silicates, such as magnesium aluminium silicate, aluminium silicate, calcium magnesium silicate, diatomaceous earth, or sodium magnesium silicate; and carbonates such as calcium carbonate or magnesium carbonate.

Among the organic fillers that may be used in the present invention, mention may be made of polyamide powders (Nylon® powders such as Nylon-6, Nylon-12, Nylon 6/12, or Polyamide-5), polytetrafluoroethylene polymer powders, polyurethane powders, polyethylene powders, acrylic polymer powders such as polymethyl methacrylate, methyl methacrylate, acrylates/ethylhexyl acrylate crosspolymer powders, copolymer of styrene and acrylic acid powders. The organic fillers that may also be used in the compositions according to the invention may also be chosen from silicone powders, such as silicone resin microbeads (Tospearl® from Toshiba), elastomeric organopolysiloxane powders, or elastomeric organopolysiloxane powders coated with silicone resin. Among these, mention may be made of polymethylsilsesquioxane, vinyl dimethicone/methicone silsesquioxane crosspolymer, trimethylsiloxysilicate, or diphenyl dimethicone/vinyl diphenyl dimethicone/silsesquioxane crosspolymer. The organic fillers may also be naturally-derived polymer powders, such as tapioca dextrin, or starch derivatives such as aluminium starch octenylsuccinate.

The fillers that may also be used in the compositions according to the invention may also be composite particles, for example composites of silica and titanium dioxide, composites of methyl methacrylate crosspolymer and polymethyl methacrylate.

The compositions according to the present invention may comprise at least one coloring agent chosen from pigments and/or nacres, and mixture thereof. The term “coloring agent” is understood to mean a white or colored particle, which may be mineral or organic and from any particle size and shape, intended to impart a visual effect to the composition. The coloring agent may represent at least 0.1% relative to the total weight of the compositions according to the invention, preferably from 0.1% to 40%, more preferably from 0.5% to 30%, better still from 1% to 20%.

The pigments may be mineral or organic particles, they may be surface-coated or uncoated. They are intended to impart color or opacity to cosmetic compositions. Mineral pigments that may be used in the present invention include metal oxides and metal hydroxides such as titanium dioxide, iron oxide, zinc oxide, zirconium oxide, aluminium oxide, chromium oxide, manganese oxide, ultramarine blue, manganese violet, iron hydroxide, magnesium hydroxide, aluminium hydroxide, chromium hydroxide, and mixture thereof. Organic pigments that may be used in the present invention include dyes and lakes such as FD&C dyes or D&C dyes, cochineal carmine.

The term “nacre” is understood to mean white or colored particles of any form, whether or not iridescent, which impart a color effect via optical interference. They may be constituted by a substrate at the surface of which is preferably deposited at least one layer of at least one material. This material may advantageously be chosen from metal oxide, organic dyestuff or mixture thereof. The substrate may be of any material, and have any shape and any particle size. When the nacre has a multilayer structure, these layers may have the same thickness or have different thickness, and they may be of the same material or of different materials.

The substrate may be selected from mica, alumina, synthetic fluorphlogopite, sericite, glass, silica, silicates such as borosilicate, or aluminosilicate. The metal oxide may be chosen from titanium dioxide, iron oxide, tin oxide, silver oxide, bismuth oxychloride, and chromium oxide. The organic dyestuff may be chosen from lakes and dyes.

The compositions according to the invention may also comprise at least one UV screening agent chosen from mineral and/or organic sunscreen agents.

Examples of the inorganic sunscreens include pigments and nanopigments formed from coated or uncoated metal oxides. Among metal oxides, mention may be made of titanium oxide, iron oxide, zinc oxide, zirconium oxide and cerium oxide nanopigments, which are all well-known as UV photoprotective agents.

Examples of organic sunscreens include dibenzoylmethane derivatives; cinnamic acid derivatives; salicylates derivatives; para-aminobenzoic acids; 0,0′-diphenylacrylate derivatives; benzophenone derivatives; benzylidenecamphor derivatives; phenylbenzimidazole derivatives; triazine derivatives; phenylbenzotriazole derivatives; anthranilic acid derivatives, and mixtures thereof. All of them may be encapsulated.

Non-limiting examples of organic filters that may be used in the present invention include those having the INCI names Benzophenone-1, Benzophenone-2, Benzophenone-3, Benzophenone-4, Benzophenone-5, Benzophenone-6, Benzophenone-8, Benzophenone-9, butyl methoxydibenzoylmethane (commercially available from HOFFMANN LA ROCHE under the trade name of Parsol 1789), octyl methoxycinnamate (commercially available from HOFFMANN LA ROCHE under the trade name of Parsol MCX), cinoxate, terephtalylidene dicamphor sulphonic acid, 3-benzylidene Camphor, Camphor Benzalkonium Methosulfate, Bis-Ethylhexyloxyphenol Methoxyphenyl Triazine, Diethylamino Hydroxybenzoyl Hexyl Benzoate, Diisopropyl Methyl Cinnamate, 1-(3,4-Dimethoxyphenyl)-4,4-Dimethyl-1,3-Pentanediene, Disodium Phenyl Dibenzimidazole Tetrasulfonate, Drometrizole trisiloxane, Ethylhexyl Dimethoxybenzylidene Dioxoimidazolidine Propionate, Ethylhexyl Dimethyl PABA, Ethylhexyl Methoxycinnamate, Ethylhexyl Salicylate, Ethylhexyl Triazone, Ferulic Acid, 4-(2-Beta-Glucopyranosiloxy) Propoxy-2-Hydroxybenzophenone, Glyceryl Ethylhexanoate Dimethoxycinnamate, Homosalate, Isoamyl p-Methoxycinnamate, Isopentyyl Trimethoxycinnamate Trisiloxane, Isopropyl Methoxycinnamate,

Menthyl Anthranilate, 4-Methylbenzylidene Camphor, Methylene Bis-Benzotriazolyl Tetramethylbutylphenol, Octocrylene, PABA, PEG-25 PABA, Pentyl Dimethyl PABA, Phenylbenzimidazole Sulfonic Acid and its salts, Polyacrylamidomethyl Benzylidene Camphor, Polysilicone-15, Potassium Phenylbenzimidazole Sulfonate, Sodium PhenylbenzimidazoleSulfonate,

TEA-Phenylbenzimidazole Sulfonate, TEA Salicylate, Terephthalylidene Dicamphor Sulfonic Acid, and mixtures thereof.

The compositions according to the present invention may also comprise additional ingredients usually used in cosmetics, such as preserving agents, cosmetic active ingredients, moisturizers, and/or fragrances.

The preserving agents that may be used include for example Ammonium silver zinc aluminium silicate, chlorophenesin, potassium sorbate, sodium dehydroacetate, phenoxyethanol, and mixture thereof.

Among the cosmetic active ingredients that may be used in the present invention, mention may be made of whitening agents, brightening agents, antioxidant agents, anti-wrinkles agents, antiseborrheic agents, plant extracts, and mixture thereof.

Non-limiting examples of active ingredients include vitamin derivatives, such as tocopheryl actetate, ascorbic acid derivatives such as ascorbyl glucoside, Niacinamide, Licorice extract, Kalanchoe Pinnata leaf extract, Vanilla Planifolia extract.

It is a matter of routine operations for a person skilled in the art to adjust the nature and amount of the additives present in the compositions according to the invention such that the desired cosmetic properties and stability properties thereof are not affected.

A cosmetic composition of the invention may be in the form of a skincare or makeup product, for example in the form of a concealer, a makeup base, a foundation, a primer. When the composition is a foundation, it further contains at least one colorant as described above, in an amount sufficient to provide additional color to or change the color of the skin.

The invention also relates concerns a process comprising a step of applying at least one layer of the composition according to the invention onto the skin, in particular the skin of the face.

The composition according to the present invention may be manufactured by the known processes generally used in cosmetics and personal care products.

### Examples

The examples which follow are used to illustrate the invention without however presenting a restrictive character. In these examples, the quantities of the ingredients compositions are given in weight percentage compared to the total weight of the composition.

In order to demonstrate the influence of nanocrystalline cellulose on the optical properties in reflection of a cosmetic composition, a series of four powders were formulated in a cosmetic base in the form of an oil-in-water emulsion. The powders were introduced at 7 wt. % into said cosmetic base thus forming four cosmetic compositions, and the fifth cosmetic composition corresponds to the reference cosmetic base without any powder. The compositions are listed in the following Table 1, wherein each ingredient is represented by the weight percentage relative to the total weight of composition.

TABLE 1
EXAMPLES

| | Ingredients: | Ex. 1 | Ex. 2 | Ex. 3 | Ex. 4 | Ex. 5 |
|---|---|---|---|---|---|---|
| | Commercial name | (inven- | (com- | (com- | (com- | (com- |
| Phase | (INCI NAME) | tive) | parative) | parative) | parative) | parative) |
| A | Demineralized Water | 66.82 | 71.85 | 66.82 | 66.82 | 66.82 |
| | (WATER) | | | | | |
| | 1,3 Butylene Glycol | 4.65 | 5 | 4.65 | 4.65 | 4.65 |
| | (BUTYLENE GLYCOL) | | | | | |
| | Glycerine 99.5% PF | 5.58 | 6 | 5.58 | 5.58 | 5.58 |
| | (GLYCERIN) | | | | | |
| | Natpure Trap TSJ | 0.08 | 0.09 | 0.08 | 0.08 | 0.08 |
| | (AQUA (WATER) & PHYTIC ACID | | | | | |
| | & SODIUM HYDROXIDE & | | | | | |
| | SODIUM CITRATE & SODIUM | | | | | |
| | BENZOATE) | | | | | |
| | Sepicide LD | 0.8 | 0.86 | 0.8 | 0.8 | 0.8 |
| | (PHENOXYETHANOL) | | | | | |
| B | Aristoflex AVC | 1.12 | 1.2 | 1.12 | 1.12 | 1.12 |
| | (AMMONIUM | | | | | |
| | ACRYLOYLDIMETHYLTAURATE/ | | | | | |
| | VP COPOLYMER) | | | | | |
| C | Xiameter PMX-200 SIL Fluid 5CS | 9.30 | 10 | 9.30 | 9.30 | 9.30 |
| | (DIMETHICONE) | | | | | |
| | NANOCRYSTALLINE CELLULOSE | 7 | — | — | — | — |
| | (CELLULOSE) | | | | | |
| | CELLULOBEADS D-10 | — | — | 7 | — | — |
| | (CELLULOSE) | | | | | |
| | COVABEAD PMMA | — | — | — | 7 | — |
| | (POLYMETHYL METHACRYLATE) | | | | | |
| | COVABEAD LH 170 | — | — | — | — | 7 |
| | (METHYL METHACRYLATE | | | | | |
| | CROSSPOLYMER) | | | | | |
| D | Alcool Export A 96°2 | 4.65 | 5 | 4.65 | 4.65 | 4.65 |
| | (ETHANOL) | | | | | |
| | total | 100 | 100 | 100 | 100 | 100 |

For making each of the examples of table 1, the following procedure was used: the ingredients of phase A were added to the flask and mixed with Rayneri at 600 rpm until homogeneous. The ingredient of phase B was added in phase A and mixed with Rayneri at 1900 rpm until gelification. Then, the ingredients from phase C were added at 1900 rpm for 10 minutes. The ingredient of phase D was then added and the mixture was homogenized with Rayneri at 1900 rpm for 5 minutes.

Protocol for Measuring In Vitro Optical Properties of Compositions

An ERICHSEN automatic draw-down instrument was used to produce 30 μm-thick films of each formula of Table 1 on 100 μm-thick PET substrates from BYK sold under reference No. 2870. The films were dried for four hours at 35° C.

Reflectance and transmittance measurements were made using a Konica Minolta CM-3600A spectrophotometer. All measurements were made four times for each sample. The values were averaged. The results are summarized in Table 2.

TABLE 2
In vitro optical parameters

| | | WHITENING | TRANSPARENCY | |
|---|---|---|---|---|
| | MATTITY | INDEX | INDEX | HAZE |
| Composition | (%) | (%) | (%) | (%) |
| Example 1 | 92.89 | 9.19 | 85.88 | 81.55 |
| (inventive) | (±0.92) | (±0.15) | (±0.20) | (±1.12) |
| Example 2 | 6.88 | 0.64 | 90.01 | 2.37 |
| (comparative) | (±1.06) | (±0.10) | (±0.10) | (±1.17) |
| Example 3 | 60.70 | 5.61 | 87.44 | 52.13 |
| (comparative) | (±0.01) | (±0.07) | (±0.08) | (±0.57) |
| Example 4 | 84.95 | 7.84 | 83.54 | 67.31 |
| (comparative) | (±0.13) | (±0.06) | (±0.66) | (±1.67) |
| Example 5 | 85.72 | 10.87 | 85.70 | 71.01 |
| (comparative) | (±0.04) | (±0.01) | (±1.05) | (±2.14) |

Values in parenthesis are the standard deviations of the raw values.

From the results in Table 2 above, it was found that example 1 yielded the highest values for haze and for mattity while exhibiting satisfactory values of transparency and whitening index.

Haze and mattity are directly related to the ability of the composition to decrease the visibility of skin imperfections such as pores, fine lines and wrinkles, and to reduce the shiny effect engendered by sebum secretion. At the same time, transparency and whitening values are directly correlated to a translucent/transparent and natural appearance of the skin.

1. A cosmetic composition comprising, in a physiologically acceptable medium, nanocrystalline cellulose, wherein the composition is topically applied onto the skin of a subject in need of the application to reduce the visibility of skin imperfections and/or to impart a matte effect on the skin.

2. The cosmetic composition according to claim 1, wherein the composition has a haze of greater than or equal to 50%, preferably greater than or equal to 60%, more preferably greater than or equal to 75%.

3. The cosmetic composition according to claim 1, wherein the composition has a mattity of greater than or equal to 70%, preferably greater than or equal to 75%, more preferably greater than or equal to 85%.

4. The cosmetic composition according to claim 1, wherein the composition has a total transmittance of greater than or equal to 70% preferably greater than or equal to 75%, more preferably greater than or equal to 85%.

5. The cosmetic composition according to claim 1, wherein the composition has a diffuse reflectance of less than or equal to 10%.

6. The cosmetic composition according to claim 1, wherein said nanocrystalline cellulose is a functionalized nanocrystalline cellulose.

7. The cosmetic composition according to claim 2, wherein said functionalized nanocrystalline cellulose is carboxylated nanocrystalline cellulose.

8. The cosmetic composition according to claim 3, wherein said carboxylated nanocrystalline cellulose is in the form of a nanocrystalline cellulose carboxylate salt.

9. The cosmetic composition according to claim 4, wherein said nanocrystalline cellulose carboxylate salt is a nanocrystalline cellulose sodium carboxylate.

10. The cosmetic composition according to claim 5, wherein said nanocrystalline cellulose sodium carboxylate is produced by the method comprising the steps of:
a) providing cellulose,
b) mixing said cellulose with a peroxide, thereby producing a reaction mixture,
c) heating the reaction mixture, and/or exposing the reaction mixture to UV radiation, and
d) salifying the reaction mixture.

11. The cosmetic composition according to claim 1, wherein said nanocrystalline cellulose has a spherical or ovoid shape.

12. The cosmetic composition according to claim 1, wherein said nanocrystalline cellulose has an average particle size of less than about 20 μm.

13. The cosmetic composition according to claim 1, wherein said nanocrystalline cellulose has an average particle size from about 2 μm to about 10 μm.

14. The cosmetic composition according to claim 1, wherein said nanocrystalline cellulose has a refractive index comprised between 1.35 and 1.6.

15. The cosmetic composition according to claim 1, wherein the composition further comprises at least one coloring agent selected from pigments and nacres and mixture thereof.

16. The cosmetic composition according to claim 1, wherein the composition further comprises at least one filler selected from organic fillers and inorganic fillers and mixtures thereof.

17. The cosmetic composition according to claim 1, wherein the composition further comprises at least one fatty phase

18. The cosmetic composition according to claim 1, wherein the composition further comprises at least one aqueous phase

19. The cosmetic composition according to claim 1, wherein the composition further comprises at least one UV screening agent selected from mineral UV screening agents and/or organic UV screening agents.

20. The cosmetic composition according to claim 1, wherein the composition further comprises at least one additional ingredient selected from preservatives, cosmetic active ingredients, moisturizers, surfactants and/or fragrances.

21. The cosmetic composition according to claim 1, wherein the composition is in the form of an oil-in-water emulsion or a water-in-oil emulsion.

22. The cosmetic composition according to claim 1, wherein the composition is in the form of a compact powder.

23. The cosmetic composition according to claim 1, wherein the composition is in the form of an anhydrous composition.

24. The cosmetic composition according to claim 1, wherein the composition is selected from the group consisting of a face powder, a foundation, a blusher, a concealer, a skin care composition, an eye shadow, a lipstick, a lip gloss, a nail enamel.

25. The cosmetic composition according to claim 1, wherein the composition is selected from the group consisting of a face powder, a foundation, a blusher, a concealer, a skin care composition.

26. A method of providing soft-focus, mattity and natural appearance to the skin comprising topically applying onto the skin of a subject in need thereof a cosmetic composition comprising nanocrystalline cellulose.

27. A method for reducing the visibility of skin imperfections and/or to imparting matte effect to the skin, comprising topically applying onto the skin of a subject in need of the application a cosmetic composition comprising nanocrystalline cellulose.
